(19) United States
(12) Patent Application Publication
(10) Pub. No.: US 2019/0167941 A1
(43) Pub. Date: Jun. 6, 2019

(54) DROWSINESS ELIMINATING DEVICE

(71) Applicant: AISIN SEIKI KABUSHIKI KAISHA, Kariya-shi (JP)

(72) Inventors: Hideaki YAMAGUCHI, Okazaki-shi (JP); Takayuki KODAMA, Otsu-shi (JP)

(73) Assignee: AISIN SEIKI KABUSHIKI KAISHA, Kariya-shi (JP)

(21) Appl. No.: 16/324,006

(22) PCT Filed: Aug. 3, 2017
(86) PCT No.: PCT/JP2017/028258

§ 371 Date: Feb. 7, 2019

(30) Foreign Application Priority Data
Aug. 12, 2016 (JP) 2016-158434

(51) Int. Cl. A61M 21/00; B60N 2/90

(52) U.S. Cl. CPC A61M 21/00; B60N 2/976; B60N 2002/981; A61M 2021/0083; A61M 2205/50; A61M 2021/0022

(57) ABSTRACT

The drowsiness eliminating device includes one of a first stimulus imparting device group including a first stimulus imparting device and a second stimulus imparting device imparting the stimulus to the first skin portion which is a skin part covering a skeletal muscle associated with a first motion illusion induced at a supplementary motor area activated by the stimulus to the tendon of latissimus dorsi and a second stimulus imparting device group including a third stimulus imparting device and a fourth stimulus imparting device imparting the stimulus to the second skin portion which is a skin part covering a skeletal muscle associated with a second motion illusion induced at a supplementary motor area activated by the stimulus to the tendon of the gluteus medius muscle and a drive control for drive-controlling one of first stimulus imparting device group and second stimulus imparting device group to impart a stimulus to the user.

### CROSS-REFERENCE TO RELATED APPLICATIONS

This application is a United States national stage application of International Application No. PCT/JP2017/028258, filed Aug. 3, 2017, which designates the United States, and claims priority to Japanese Patent Application No. 2016-158434, filed Aug. 12, 2016, and the entire contents of each of the above applications are hereby incorporated herein by reference in entirety.

### BACKGROUND

### Technical Field

This application relates to a drowsiness eliminating device.

### Background Art

As one type of drowsiness eliminating devices, a drowsiness eliminating device disclosed in Patent Literature 1 has been known. As shown in FIG. 2 of the Patent Literature 1, the drowsiness eliminating device includes a danger detecting means which includes a vehicle lane deviating predicting and detecting means and/or a driver drowsiness detecting means and an alarm motor control means which generates an alarm vibration to an electric seat by rotating a motor in normal/reverse directions which operates the electric motor, by outputting normal/reverse rotation signal upon receipt of a detecting signal from the danger detecting means. Thus, the drowsiness eliminating device generates an alarm to the driver of the vehicle by vibration, utilizing a motor which is prepared in advance for operating each part of the electric seat.

Further, as another type of the drowsiness eliminating devices, a drowsiness eliminating device disclosed in Patent Literature 2 has been known. As shown in FIG. 1 of the Patent Literature 2, according to the drowsiness eliminating device disclosed therein, when a driver D becomes drowsy, the air bag 3 expands and imparts a pressing stimulus (baresthesia) as an awakening stimulus to the driver D's musculus erector spinae and the biomedical measurement device 8 measures the muscle potential as a parameter indicating the activity state of the musculus erector spinae. Then, a wakening state maintaining ECU 11 controls the strength of the baresthesia which is the awakening stimulus to be imparted to the musculus erector spinae by the air bag 3 based on the measured muscle potential to a level that the brain cell of the driver D is activated. In addition, the drowsiness eliminating device is configured such that the vibration body 7 imparts vibration stimulus to the musculus erector spinae.

### RELATED ART LITERATURE

### Patent Literature

Patent Literature 1 JP 2008-260444 A

Patent Literature 2 JP2010-172541 A

### SUMMARY

### Technical Problem(S)

According to the drowsiness eliminating device disclosed in the Patent Literature 1, a vibration is applied to the electric seat and the vibration is used as an alarm to the driver of the vehicle to be able to inform the driver of a danger. However, it is not sufficient for the driver of the vehicle to eliminate drowsiness, merely informing the driver of the danger.

Further, according to the drowsiness eliminating device disclosed in the Patent Literature 2, the air bag 3 expands and imparts baresthesia stimulus which is the awakening stimulus to the driver D's musculus erector spinae to thereby impart stimulus to the driver D. However, the part of the body to which the stimulus is imparted in this drowsiness eliminating device is not a proper portion and the driver would not sufficiently eliminate the drowsiness.

The present application has been made to eliminate the aforementioned problem and the objective of the application is to provide a drowsiness eliminating device which can impart stimulus to a part where more awakening effect can be achieved.

### Solution to Problem(S)

In order to solve the above problem(s), the drowsiness eliminating device according to an aspect of the application includes at least one of a first stimulus imparting device group including a first stimulus imparting device provided on a chair such that the tendon of latissimus dorsi of a user of the chair faces when the user sits on the chair and imparting a stimulus to the tendon of latissimus dorsi of the user and a second stimulus imparting device provided on the chair facing a first skin portion when the user sits on the chair and imparting the stimulus to the first skin portion of the user which is a skin part covering a skeletal muscle which is associated with a first motion illusion induced at a supplementary motor area activated by the stimulus to the tendon of latissimus dorsi and which is a cooperative muscle of the latissimus dorsi and a second stimulus imparting device group including a third stimulus imparting device provided on the chair such that the tendon of gluteus medius muscle of the user of the chair faces when the user sits on the chair and imparting a stimulus to the tendon of gluteus medius muscle of the user and a fourth stimulus imparting device provided on the chair facing a second skin portion when the user sits on the chair and imparting the stimulus to the second skin portion of the user which is a skin part covering a skeletal muscle which is associated with a second motion illusion induced at a supplementary motor area activated by the stimulus to the tendon of gluteus medius muscle and which is a cooperative muscle of the gluteus medius muscle and a drive control device which includes a driving control portion which drive-controls said at least one of the first stimulus imparting device group and the second stimulus imparting device group to impart stimulus to the user of the chair.

According to such a configuration, the drive control device imparts stimulus to the tendon of latissimus dorsi of the user by the first stimulus imparting device and to the first skin portion of the user by the second stimulus imparting device, and/or the drive control device imparts stimulus to the tendon of gluteus medius muscle of the user by the third stimulus imparting device and to the second skin portion of the user by the fourth stimulus imparting device. Accordingly, in addition to properly stimulating tendon of latissimus dorsi and/or gluteus medius muscle which are the parts highly effective to awaken the user, it is possible to properly stimulate a first skin portion which is a skin part covering the skeletal muscle that is a cooperative muscle of the latissimus dorsi of the user of the chair which is associated with a first motion illusion which is induced at the supplementary motor area activated by the stimulus to the tendon of latissimus dorsi and/or properly stimulate a second skin portion which is a skin part covering the skeletal muscle that is a cooperative muscle of the gluteus medius muscle of the user which is associated with a second motion illusion which is induced at the supplementary motor area activated by the stimulus to the tendon of gluteus medius muscle. Thus, by simulating two portions which awakening effects are mutually correlated to thereby achieve a higher awakening effect. Accordingly, the drowsiness eliminating device can properly stimulate the portions where the awakening effect is high.

### BRIEF DESCRIPTION OF THE DRAWINGS

FIG. 1 is a schematic view showing a concept of a first embodiment of a drowsiness eliminating device according to the present application;

FIG. 2 is an upper plan view showing the drowsiness eliminating device indicated in FIG. 1 and indicating a state that the vibration portion of the drowsiness eliminating device is stored;

FIG. 3 is an upper plan view showing the drowsiness eliminating device indicated in FIG. 1 and indicating a state that the vibration portion of the drowsiness eliminating device is in contact with the user;

FIG. 4 is a view showing a drowsiness generation suppression effect of various types of skeletal muscles;

FIG. 5 is a view showing the drowsiness generation suppression effect of tendon portion and the abdominal part of the same skeletal muscle;

FIG. 6 is a correlation of the nerve activity value of the supplementary motor area and the drowsiness eliminating effect;

FIG. 7 shows a bar chart at left side showing the nerve activity value of the supplementary motor area when only the gluteus medius muscle is stimulated with a predetermined stimulus, whereas a bar chart at the right side showing the nerve activity value of the supplementary motor area when both of the gluteus medius muscle and the femur outside portion which corresponds to the second skin portion are stimulated with a predetermined stimulus;

FIG. 8 is a block diagram indicating the control device shown in FIG. 1;

FIG. 9 is a view indicating a drowsiness generation suppression effect per frequency of vibration stimulus;

FIG. 10 is a view indicating a drowsiness generation suppression effect per time of imparting vibration stimulus;

FIG. 11 is a view indicating a drowsiness eliminating effect when a vibration stimulus is imparted to the latissimus dorsi;

FIG. 12 is a view indicating a drowsiness eliminating effect when a vibration stimulus is imparted to the gluteus medius muscle;

FIG. 13 is a view indicating the drowsiness eliminating effect when a vibration stimulus is imparted to only the tendon of the gluteus medius muscle;

FIG. 14 is a view indicating a drowsiness eliminating effect when a vibration stimulus is imparted to both of the tendon of the gluteus medius muscle and the skin portion (second skin portion) of femur outside portion;

FIG. 15 is a view indicating a drowsiness eliminating effect when a vibration stimulus is imparted to only the skin portion (second skin portion) of femur outside portion;

FIG. 16 is a flowchart of a control program which is executed by the control device shown in FIG. 1; and

FIG. 17 is a schematic view showing an outline of the drowsiness eliminating device according to the second embodiment of the application.

### DETAILED DESCRIPTION

### First Embodiment

Hereinafter, a first embodiment of a drowsiness eliminating device according to the present application will be explained. As shown in FIG. 1, a drowsiness eliminating device 10 is installed in a vehicle A. The drowsiness eliminating device 10 is provided at a vehicle seat 20 that is a driver's seat of the vehicle A. The vehicle seat 20 is a chair on which the driver M sits as a user. The vehicle seat 20 includes a seat cushion 21 and a seat back 22.

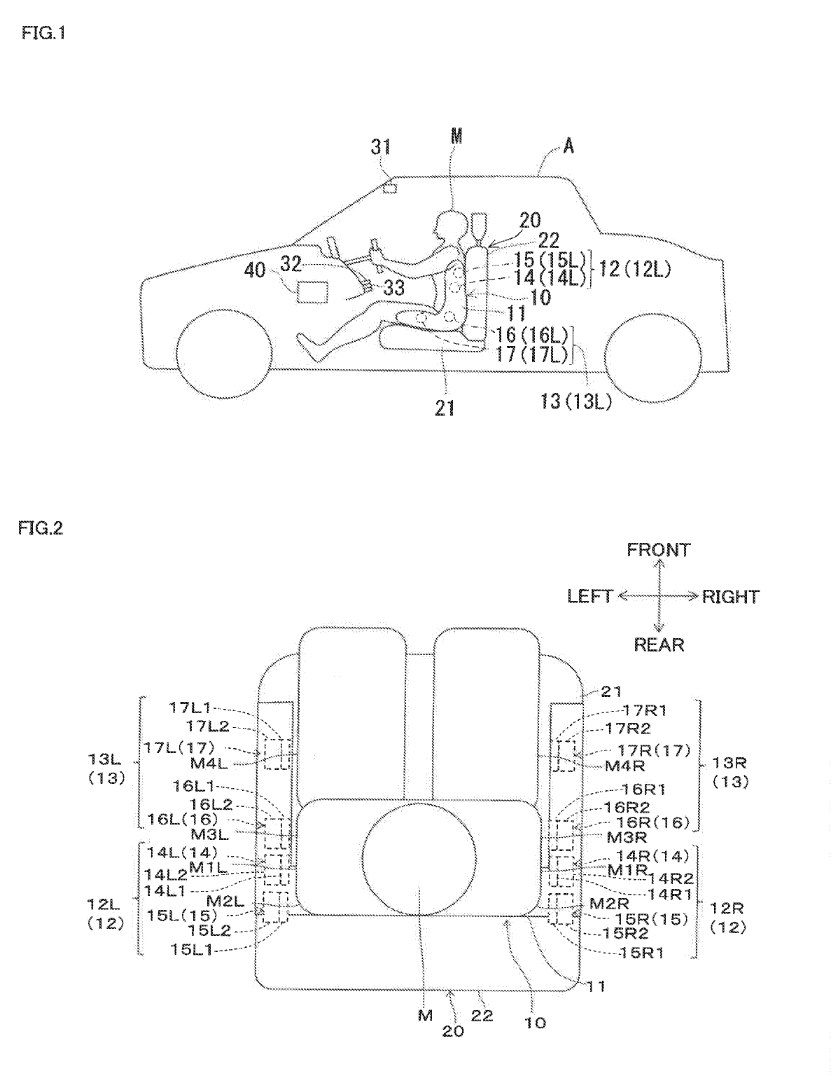

The drowsiness eliminating device 10 includes, as shown in FIGS. 1 and 2, a main body 11, a first stimulus imparting device group 12 provided at the main body 11, a second stimulus imparting device group 13 provided at the main body 11 and a control device 40 which controls the first stimulus imparting device group 12 and the second stimulus imparting device group 13.

It is noted here that the control device 40 may be installed at the vehicle body side (for example, on the vehicle seat 20 or vehicle frame, etc.). Still further, the control device 40 may be stored in the main body 11.

The main body 11 is formed separately from the vehicle seat 20 and can be detachably assembled with the vehicle seat 20. The main body 11 is formed in shape of a bucket seat for the driver M free to be seated. The bucket seat shape is shaped such that the height of the right and left ends thereof is formed extremely high compared to a normal type of vehicle seat so as to deeply embrace the femur, hips, and shoulder of the driver M. The main body 11 is structured to cover or shroud the flank, waist, and femur of the body side of the driver M (user).

The first stimulus imparting device group 12 includes a first stimulus imparting device 14 and a second stimulus imparting device 15. The first stimulus imparting device group 12 includes a right side first stimulus imparting device group 12R which is arranged at the right side and a left side first stimulus imparting device group 12L arranged at the left side. The right side first stimulus imparting device group 12R includes a right side first stimulus imparting device 14R and a right side second stimulus imparting device 15R. The left side first stimulus imparting device group 12L includes a left side first stimulus imparting device 14L and a left side second stimulus imparting device 15L.

The first stimulus imparting device 14 is provided at the main body 11 and serves as a device which imparts stimulus to the tendon of the latissimus dorsi of the driver M. The first stimulus imparting device 14 is provided at the vehicle seat 20 via the main body 11. The first stimulus imparting device 14 includes the right side first stimulus imparting device (right side latissimus dorsi stimulus imparting device) 14R which imparts stimulus to the right side latissimus dorsi and the left side first stimulus imparting device (left side latissimus dorsi stimulus imparting device) 14L which imparts stimulus to the left side latissimus dorsi. It is preferable to arrange the right side first stimulus imparting device 14R in the vicinity of a portion of the main body 11 facing the right side tendon M1R of the latissimus dorsi of the driver M when the driver M is seated on the main body 11. It is also preferable to arrange the left side first stimulus imparting device 14L in the vicinity of a portion of the main body 11 facing the left side tendon M1L of the latissimus dorsi of the driver M when the driver M is seated on the main body 11.

The second stimulus imparting device 15 is provided at the main body 11 and serves as a device which imparts stimulus to the first skin portion of the driver M. The first skin portion is a skin part covering the skeletal muscle associated with a first motion illusion which is induced at the supplementary motor area (a portion of brain) activated by the stimulus to the tendon of latissimus dorsi and which is a cooperative muscle of the latissimus dorsi. In more detail, the first motion illusion is a motion illusion that the arm moves upward and the first skin potion is a skin portion (skin portion located in the vicinity of skeletal muscle (for example, serratus anterior muscle) which covers skeletal muscle (for example, serratus anterior muscle) associated with the first motion illusion and is a cooperative muscle of the latissimus dorsi. The first skin portion is a skin area, for example, from the inferior angle of scapula to the armpit portion. It is noted that the “cooperative muscle” is a muscle which helps the work of protagonist muscle that is the main muscle for action, when a human makes a motion.

In the motion that the arm moves downward (contract of latissimus dorsi), the latissimus dorsi works as the protagonist muscle and the serratus anterior muscle works as the cooperative muscle. When a stimulus (for example, vibration stimulus) is imparted to the tendon of latissimus dorsi, the activity of muscle spindle becomes high and such information is transmitted to the centrum system (brain) and accompanied by this activity, the motion illusion (first motion illusion) is induced. The information from the muscle spindle is the information that “the muscle is expanded” and this is erroneously recognized (illusion) that the latissimus dorsi is expanding, i.e., the arm is moving upward. On the other hand, when the arm is actually moved upward, the latissimus dorsi is expanded and the skin in the vicinity thereof (skin portion covering the cooperative muscle) also expands and such expansion information is transmitted to the centrum. On the contrary, when a stimulus is imparted to the latissimus dorsi, since the arm is not actually moving (illusion only), the information from the skin portion covering the cooperative muscle is not transmitted to the centrum by the stimulus imparted only to the tendon of latissimus dorsi. Therefore, in addition to the stimulus to the tendon of latissimus dorsi, by imparting the stimulus to the skin portion associated with the motion illusion, the motion illusion (activity of supplementary motor area) can be heightened. Thus, the tendon of latissimus dorsi and the skin portion which covers the serratus anterior muscle as the cooperative muscle of latissimus dorsi are two parts correlated in the first motion illusion.

The second stimulus imparting device 15 is provided at the vehicle seat 20 via the main body 11. The second stimulus imparting device 15 includes the right side second stimulus imparting device (right side first skin portion stimulus imparting device) 15R which imparts stimulus to the right side first skin portion and the left side second stimulus imparting device (left side first skin portion stimulus imparting device) 15L which imparts stimulus to the left side first skin portion. It is preferable to arrange the right side second stimulus imparting device 15R in the vicinity of a portion of the main body 11 facing the right side first skin portion M2R of the driver M when the driver M is seated on the main body 11. It is also preferable to arrange the left side second stimulus imparting device 15L in the vicinity of a portion of the main body 11 facing the left side first skin portion M2L of the driver M when the driver M is seated on the main body 11.

The second stimulus imparting device group 13 includes a third stimulus imparting device 16 and a fourth stimulus imparting device 17. The second stimulus imparting device group 13 includes a right side second stimulus imparting device group 13R which is arranged at the right side and a left side second stimulus imparting device group 13L arranged at the left side. The right side second stimulus imparting device group 13R includes a right side third stimulus imparting device 16R and a right side fourth stimulus imparting device 17R. The left side second stimulus imparting device group 13L includes a left side third stimulus imparting device 16L and a left side fourth stimulus imparting device 17L.

The third stimulus imparting device 16 is provided at the main body 11 and serves as a device which imparts stimulus to the tendon of the gluteus medius muscle of the driver M. The third stimulus imparting device 16 is provided at the vehicle seat 20 via the main body 11. The third stimulus imparting device 16 includes the right side third stimulus imparting device (right side gluteus medius muscle stimulus imparting device) 16R which imparts stimulus to the right side gluteus medius muscle and the left side third stimulus imparting device (left side gluteus medius muscle stimulus imparting device) 16L which imparts stimulus to the left side gluteus medius muscle. It is preferable to arrange the right side third stimulus imparting device 16R in the vicinity of a portion of the main body 11 facing the right side tendon M3R of the gluteus medius muscle of the driver M when the driver M is seated on the main body 11. It is also preferable to arrange the left side third stimulus imparting device 16L in the vicinity of a portion of the main body 11 facing the left side tendon M3L of the gluteus medius muscle of the driver M when the driver M is seated on the main body 11.

The fourth stimulus imparting device 17 is provided at the main body 11 and serves as a device which imparts stimulus to the second skin portion of the driver M. The second skin portion is a skin part that covers the skeletal muscle that is associated with the second motion illusion induced at the supplementary motor area (a part of brain) activated by the stimulus imparted to the tendon of the gluteus medius muscle and is a cooperative muscle of the gluteus medius muscle. In more detail, the second motion illusion is an illusion that the femur portion moves inner side and the second skin potion is a skin part (skin part in the vicinity of skeletal muscle, for example, vastus lateralis that is a cooperative muscle of the gluteus medius muscle) covering the skeletal muscle that is associated with a second motion illusion and which is a cooperative muscle of the gluteus medius muscle. The second skin portion is a skin area for example, femur outside portions.

In the motion that the femur portion moves outward (contract of gluteus medius muscle), the gluteus medius muscle works as the protagonist muscle and the vastus lateralis muscle works as the cooperative muscle. When a stimulus (for example, vibration stimulus) is imparted to the tendon of gluteus medius muscle, the activity of muscle spindle becomes high and such information is transmitted to the centrum system (brain) and accompanied by this activity, the motion illusion (second motion illusion) is induced. The information from the muscle spindle is the information that “the muscle is expanded” and this is erroneously recognized (illusion) that the gluteus medius muscle is expanded, i.e., the femur portion is moved inner side. On the other hand, when the femur portion is actually moved inward, the gluteus medius muscle is expanded and the skin in the vicinity thereof (skin portion covering the cooperative muscle) also expands and such expansion information is transmitted to the centrum. On the contrary, when a stimulus is imparted to the gluteus medius muscle, since the femur portion is not actually moving (illusion only), the information from the skin portion covering the cooperative muscle is not transmitted to the centrum by the stimulus imparted only to the tendon of gluteus medius muscle. Therefore, in addition to imparting stimulus to the tendon of gluteus medius muscle, by imparting the stimulus to the skin portion associated with the motion illusion, the motion illusion (activity of supplementary motor area) can be heightened. Thus, the tendon of gluteus medius muscle and the skin portion which covers the vastus lateralis muscle as the cooperative muscle of the gluteus medius muscle are two parts correlated in the second motion illusion.

The fourth stimulus imparting device 17 is provided at the vehicle seat 20 via the main body 11. The fourth stimulus imparting device 17 includes the right side fourth stimulus imparting device (right side second skin portion stimulus imparting device) 17R which imparts stimulus to the right side second skin portion and the left side fourth stimulus imparting device (left side second skin portion stimulus imparting device) 17L which imparts stimulus to the left side second skin portion. It is preferable to arrange the right side fourth stimulus imparting device 17R in the vicinity of a portion of the main body 11 facing the right side second skin portion M4R of the driver M when the driver M is seated on the main body 11. It is also preferable to arrange the left side fourth stimulus imparting device 17L in the vicinity of a portion of the main body 11 facing the left side second skin portion M4L of the driver M when the driver M is seated on the main body 11.

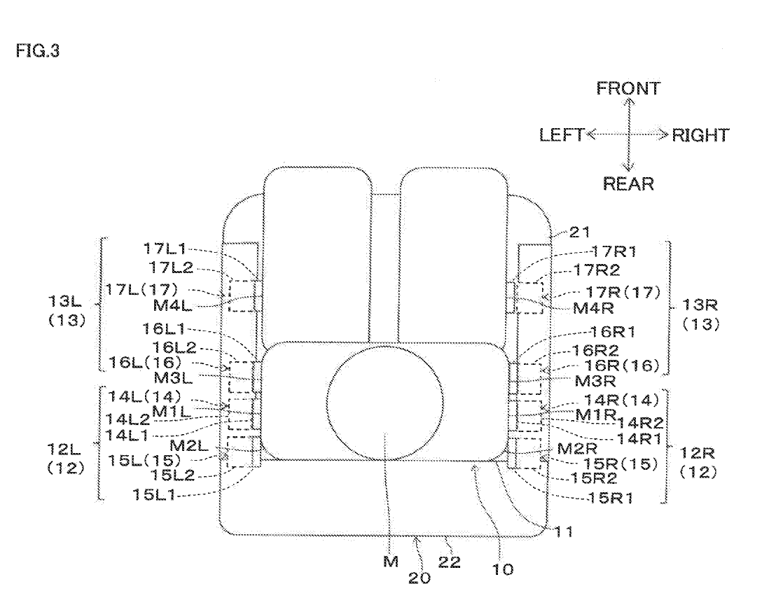

The right side first stimulus imparting device 14R includes, as shown in FIGS. 2 and 3, a vibration portion 14R1 which imparts stimulus (vibration) to the tendon M1R of the right side latissimus dorsi by contacting therewith and a moving portion 14R2 which moves the vibration portion 14R1 in advancing and retreating directions relative to the driver M. The base portion of the moving portion 14R2 is fixed to the main body 11 and the tip end of the moving portion 14R2 is fixed to the vibration portion 14R1. The vibration portion 14R1 stores a motor therein (not shown) and is configured to generate a vibration by converting the rotational movement of the motor into the linear motion. It is noted that the vibration portion 14R1 may be configured to house a linear motor therein to generate a vibration by the linear motion of the output shaft of the motor. The moving portion 14R2 may be configured to be an air bag type which contracts or expands in response to the supply or discharge of the air from an air pump, or may be configured to be a combination of link mechanism and motor.

Similar to the right side first stimulus imparting device 14R, the left side first stimulus imparting device 14L includes a vibration portion 14L1 which imparts stimulus (vibration) to the tendon M1L of the left side latissimus dorsi by contacting therewith and a moving portion 14L2 which moves the vibration portion 14L1 in advancing and retreating directions.

Similar to the right side first stimulus imparting device 14R, the right side second stimulus imparting device 15R includes a vibration portion 15R1 which imparts stimulus (vibration) to the right side first skin portion M2R by contacting therewith and a moving portion 15R2 which moves the vibration portion 15R1 in advancing and retreating directions.

Similar to the right side first stimulus imparting device 14R, the left side second stimulus imparting device 15L includes a vibration portion 15L1 which imparts stimulus (vibration) to the left side first skin portion M2L by contacting therewith and a moving portion 15L2 which moves the vibration portion 15L1 in advancing and retreating directions.

Similar to the right side first stimulus imparting device 14R, the right side third stimulus imparting device 16R includes a vibration portion 16R1 which imparts stimulus (vibration) to the right side tendon M3R of gluteus medius muscle by contacting therewith and a moving portion 16R2 which moves the vibration portion 16R1 in advancing and retreating directions. Similar to the right side first stimulus imparting device 14R, the left side third stimulus imparting device 16L includes a vibration portion 16L1 which imparts stimulus (vibration) to the left side tendon M3L of gluteus medius muscle by contacting therewith and a moving portion 16L2 which moves the vibration portion 16L1 in advancing and retreating directions.

Similar to the right side first stimulus imparting device 14R, the right side fourth stimulus imparting device 17R includes a vibration portion 17R1 which imparts stimulus (vibration) to the right side second skin portion M4R by contacting therewith and a moving portion 17R2 which moves the vibration portion 17R1 in advancing and retreating directions.

Similar to the right side first stimulus imparting device 14R, the left side fourth stimulus imparting device 17L includes a vibration portion 17L1 which imparts stimulus to the left side second skin portion M4L by contacting therewith and a moving portion 17L2 which moves the vibration portion 17L1 in advancing and retreating directions.

It is noted that the drowsiness eliminating device 10 may be configured to be housed in the vehicle seat 20.

Further, the strength (magnitude) of stimulus imparted by the second stimulus imparting device 15 may be preferably set to be smaller than the strength of the stimulus imparted by the first stimulus imparting device 14. The strength (magnitude) of stimulus imparted by the fourth stimulus imparting device 17 may be preferably set to be smaller than the strength of the stimulus imparted by the third stimulus imparting device 16.

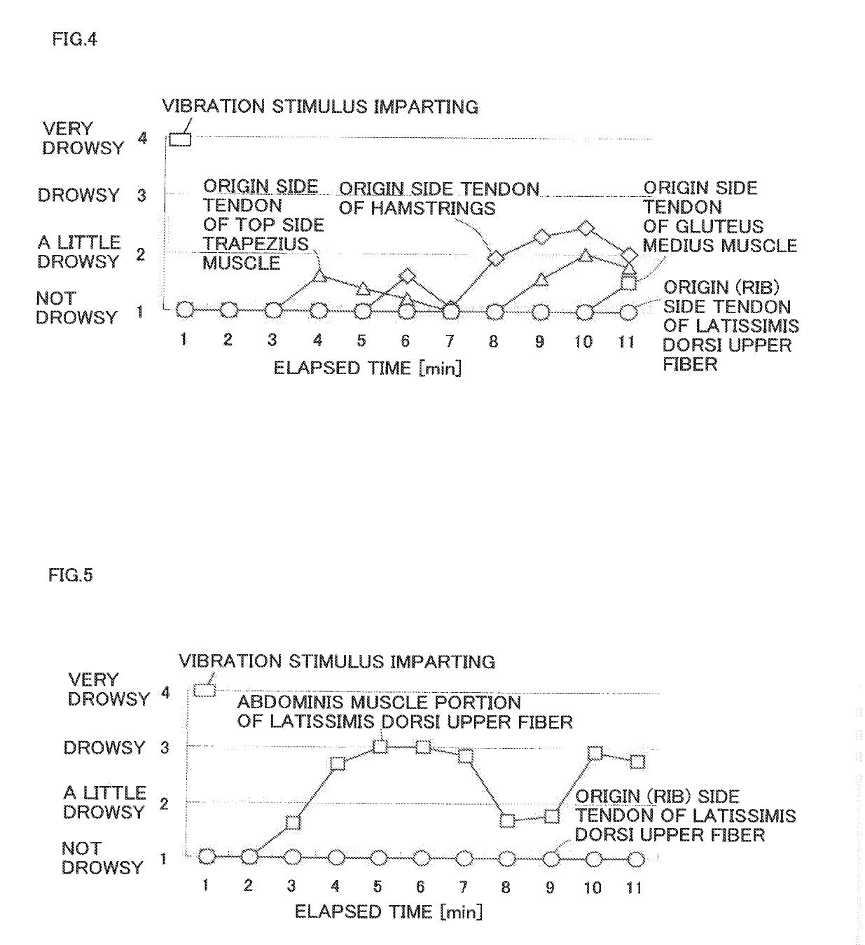

The reason why the stimulus is imparted to a particular portion (i.e., the tendon of latissimus dorsi or tendon of gluteus medius muscle) will be explained hereinafter. The inventors of the application found by experimental work that a tendon of a particular skeletal muscle (including muscle tendon junction) has a high awakening effect. Such particular skeletal muscle is the tendon of latissimus dorsi and tendon of gluteus medius muscle. FIG. 4 indicates an experimental result that a vibration stimulus is imparted to a plurality of tendons of skeletal muscles when a person in test is at a first drowsiness level where the person does not feel drowsy. The horizontal axis indicates the time between the imparting stimulus is started and a certain time period passed therefrom and the vertical axis indicates the level of drowsiness.

The vibration stimulus is the same stimulus that is continuously imparted for thirty seconds with the frequency of 100 Hz. The drowsiness level is measured by changing the skeletal muscle of the person in test to which the stimulus is imparted. The drowsiness level is defined by self-assessment of the subject person or may be defined by measurement. The white square indicates the change of drowsiness level when the stimulus is imparted to the origin side tendon of gluteus medius muscle, the diamond indicates the change of drowsiness level when the stimulus is imparted to the origin side tendon of hamstrings, the circle indicates the change of drowsiness level when the stimulus is imparted to the origin side (rib) tendon of upper side fiber latissimus dorsi and the triangle indicates change of drowsiness level when the stimulus is imparted to the origin side tendon of top side trapezius muscle. It is noted that the hamstrings is a collective name of muscles at lower limbs back side, such as biceps femoris, semimembranosus muscle and semitendinosus, etc.

Apparent from the experimental result, in the hamstrings and the trapezius muscle, the drowsiness level becomes great to the drowsiness side from the early timing. On the contrary, in the latissimus dorsi and tendon of gluteus medius muscle, the first drowsiness level that is the non-drowsy level is maintained for a relatively long time period. Accordingly, the drowsiness suppression effect is high at the latissimus dorsi and tendon of gluteus medius muscle, i.e., a high awakening effect can be maintained thereby.

It is noted that a muscle spindle exists in the skeletal muscle. The muscle spindle serves as a sensor which senses the state of muscle and the information that the muscle spindle is being activated is transmitted to the brain stem reticular formation to raise the activity level of the brain (awakening level is raised). Further, it is noted that the latissimus dorsi and tendon of gluteus medius muscle are the “type I” fiber which includes many muscle spindles and are arranged such that a plurality of skeletal muscles are not overlapped one another. Accordingly, each of these portions is not influenced by another skeletal muscle which responsiveness is different. Thus, a stable effect can be achieved thereby.

Further, the inventors found by experimental work that among the latissimus dorsi and gluteus medius muscle, the tendon portion is more effective than the abdominis muscle portion. FIG. 5 indicates the experimental work result that a vibration stimulus is imparted to the latissimus dorsi and tendon of gluteus medius muscle of the same skeletal muscle when a person in test is at a first drowsiness level where the person does not feel drowsy. The horizontal axis indicates the time between the imparting stimulus is started and a certain time period passed therefrom and the vertical axis indicates the level of drowsiness.

The vibration stimulus is the same stimulus that is continuously imparted for thirty seconds with the frequency of 100 Hz. The drowsiness level is measured by changing the skeletal muscles of the person in test to which the stimulus is imparted. The drowsiness level is defined by self-assessment of the subject person or may be defined by measurement. The white square indicates the change of drowsiness level when the stimulus is imparted to the abdominis muscle portion of upper side fiber latissimus dorsi muscle and the circle indicates the change of drowsiness level when the stimulus is imparted to the origin side (rib) tendon of upper side fiber latissimus dorsi.

Apparent from the experimental result, in the abdominis muscle portion, the drowsiness level becomes great to the drowsiness side from the early timing. To this, in the origin side tendon, the first drowsiness level that is the non-drowsy level is maintained for a relatively long time period. Accordingly, the drowsiness generation suppression effect is high at the tendon portion, i.e., a high awakening effect can be maintained.

Further, the inventors of the application found that the awakening effect is higher in the case where the stimulus is imparted to the first skin portion and the second skin portion in addition to the imparting the stimulus to the tendon of latissimus dorsi and tendon of gluteus medius muscle, than in the case where the stimulus is only imparted to the tendon of latissimus dorsi and tendon of gluteus medius muscle.

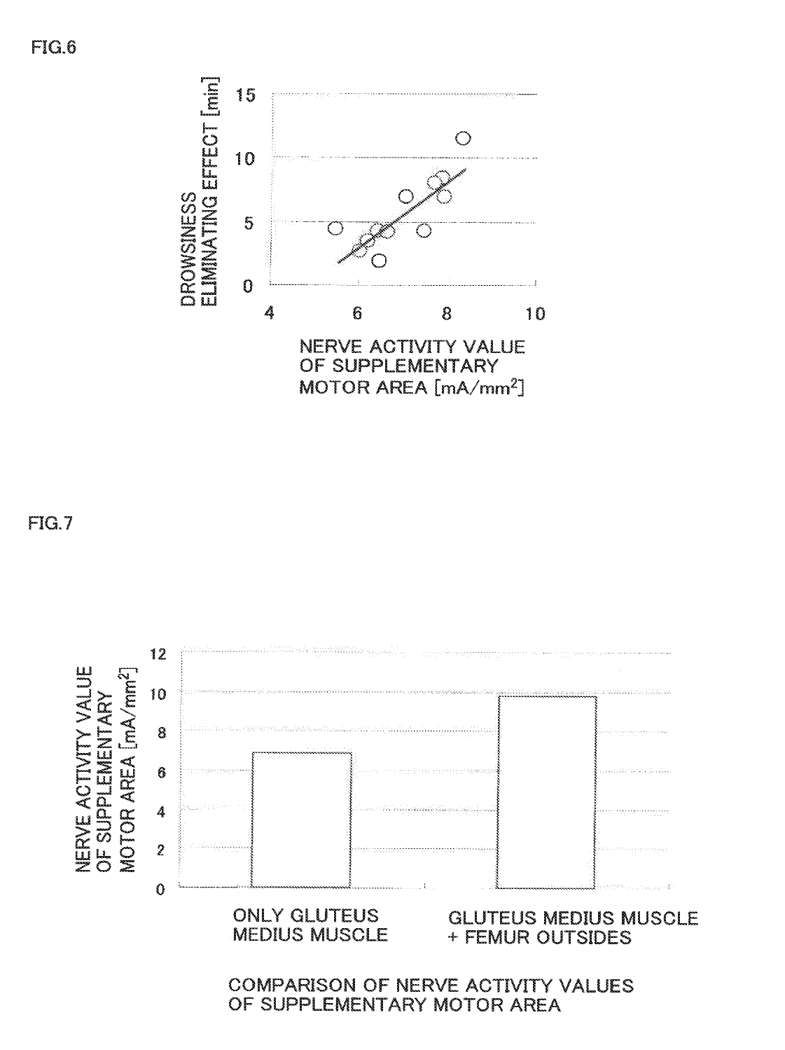

As explained above, when the stimulus is imparted to the tendon of latissimus dorsi and tendon of gluteus medius muscle, the muscle spindle is activated to activate the supplementary motor area. Under this state, the motion illusion has been induced in the brain. Further, a nerve projection is projected to the brain stem reticular formation which is associated with the brain awakening from the supplementary motor area and thus the activity of the supplementary motor area heightens the stimulus to the brain stem reticular formation. Thus, the drowsiness can be eliminated. As shown in FIG. 6, the larger the nerve activity value of the supplementary motor area, the higher the drowsiness eliminating effect becomes. In other words, the stronger the supplementary motor area is activated, the higher the drowsiness eliminating effect becomes.

On the other hand, as explained above, there is a close correlation between the activity of the supplementary motor area and the induction of the motion illusion corresponding to the supplementary motor area. In other words, the larger the degree of induction of the motion illusion corresponding to the supplementary motor area, the higher the drowsiness eliminating effect becomes. Further, the stronger the supplementary motor area is activated and at the same time the larger the degree of induction of the motion illusion corresponding to the supplementary motor area, the higher the drowsiness eliminating effect becomes. The reason is that since the supplementary motor area is strongly activated, more effective drowsiness eliminating effect can be achieved.

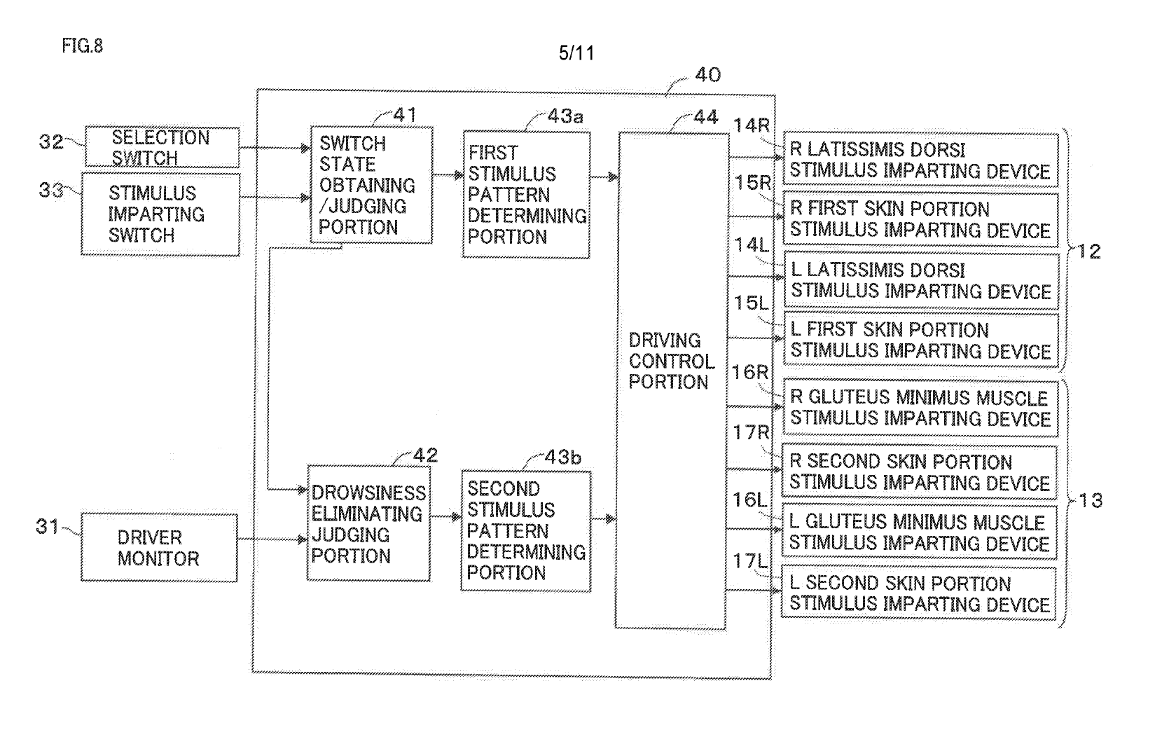

This can be apparent from the experimental data shown in FIG. 7. The bar graph at the left side of the drawing indicates the nerve activity value of the supplementary motor area when only the tendon portion of the gluteus medius muscle receives a predetermined stimulus. The bar graph at the right side of the drawing indicates the nerve activity value of the supplementary motor area when both tendon portion of the gluteus medius muscle and the femur outside portions that correspond to the second skin portion, receive a predetermined stimulus. As apparent from FIG. 7, the nerve activity value of the supplementary motor area becomes increased when both tendon portion of the gluteus medius muscle and femur outside portions that correspond to the second skin portion, receive a predetermined stimulus, compared to the case when only the tendon portion of the gluteus medius muscle receives a predetermined stimulus. Since the supplementary motor area is strongly activated, more effective drowsiness eliminating effect can be achieved.

As shown in FIG. 8, the driver monitor 31, the selection switch 32 and the stimulus imparting switch 33 are electrically connected to the control device 40.

The driver monitor 31 is installed in the vehicle A at the inside thereof (such as dashboard, or rearview mirror), as shown in FIG. 1. The driver monitor 31 serves as a device for watching the driver M and is formed by, for example, a camera, etc. The driver monitor 31 detects whether the driver M is drowsy or not from the facial expression of the driver M which expression is image-taken by the camera. For example, when the increase of the number of yawning, the increase (or decrease) of the number of blinking or a closing state of eyelid is recognized, the driver monitor 31 detects that the drowsiness exists. The detection result is outputted to the control device 40. It is noted here that in regard to the driver monitor 31, it may be configured such that the camera in the driver monitor 31 takes the image of the driver M and the expression information of the driver's image is outputted to the control device 40 and the control device 40 judges whether the driver M is drowsy or not based on the expression information.

The selection switch 32 is a switch used for selecting whether a support for eliminating drowsiness is necessary or not and the selection result is outputted to the control device 40. The support for eliminating drowsiness is a control which eliminates the drowsiness by imparting a stimulus to the driver M when the driver M feels drowsy (the third drowsiness level).

The drowsiness level is set to four stage levels, the first drowsiness level which is a non-drowsy level, the second drowsiness level which is slightly drowsy level, the third drowsiness level which is a drowsy level and the fourth drowsiness level which is a very drowsy level.

The stimulus imparting switch 33 is a switch for imparting a stimulus to the driver M on the request by the driver M. The state of ON/OFF of the switch 33 is outputted to the control device 40. When the stimulus imparting switch 33 is turned ON, each of the stimulus imparting device groups 12R, 12L, 13R and 13L is driven to impart the stimulus to the driver M with a predetermined stimulus pattern. It may be configured that a stimulus is imparted to the driver M each time the stimulus imparting switch 33 is turned ON. Alternatively, it may be configured that a stimulus is imparted to the driver M per every predetermined time period while the stimulus imparting switch 33 is being turned ON.

The control device 40 includes, as shown in FIG. 8, a switch state obtaining/judging portion 41, a drowsiness eliminating judging portion 42, each stimulus pattern determining portion 43a, 43b and a driving control portion 44.

When the selection switch 32 selects any one of the drowsiness generation suppression support, the drowsiness eliminating support or non-selection state, the switch state obtaining/judging portion 41 obtains the selection result from the selection switch 32 and judges whether the switch state is any of the selected support state and the non-selected state selected by the selection switch 32. Further, the switch state obtaining/judging portion 41 obtains the ON/OFF state of the stimulus imparting switch 33 from the stimulus imparting switch 33 and judges whether the state of the stimulus imparting switch 33 is ON or OFF.

The drowsiness eliminating judging portion 42 judges whether or not it is necessary to eliminate the drowsiness (drowsiness elimination) of the driver M sitting on the vehicle seat 20 (drowsiness eliminating judgement). The drowsiness eliminating judging portion 42 executes the drowsiness elimination judgement when the judgement result that the drowsiness eliminating support is selected by the selection switch 32 is obtained from the switch state obtaining/judging portion 41.

The drowsiness eliminating judging portion 42 obtains the detection result of the driver monitor 31 (whether the driver M is drowsy or not) from the driver monitor 31. The drowsiness eliminating judging portion 42 judges that it is necessary to eliminate the drowsiness of the driver M when the drowsiness eliminating judging portion 42 obtains the detection result that the driver M is drowsy. It is noted that the driver monitor 31 detects that the driver M is drowsy when the facial expression of the driver M changes, such as an increase of the number of yawning, an increase (or decrease) of the number of blinking or a closing state of eyelid.

It is noted that the facial expression change of the judgement subject may be configured to be selectable by the driver M. Further, it may be possible to configure that the facial expression change before and after the stimulus imparting switch 33 is ON is memorized in advance and derives facial expression changes specific to the driver M. Then, such specific facial expression changes are used for judgement of expression change of the subject.

When the first stimulus pattern determining portion 43a obtains the judgement result that the stimulus imparting switch 33 is turned ON from the switch state obtaining/judging portion 41, the first stimulus pattern determining portion 43a determines the first stimulus pattern. The first stimulus pattern is the base stimulus pattern. It is preferable to set the frequency from 90 to 110 Hz and to set the one time continuous imparting time from 20 to 40 seconds for the first stimulus patterns of the first stimulus imparting device 14 and the third stimulus imparting device 16 among the first stimulus patterns. It is also preferable to set the frequency to be 100 Hz and is preferable to set the continuous imparting time to be 30 seconds.

Further, it is preferable to set the frequency from 40 to 60 Hz and to set the one time continuous imparting time from 20 to 40 seconds for the first stimulus patterns of the second stimulus imparting device 15 and the fourth stimulus imparting device 17 among the first stimulus patterns when the stimulus imparted is the vibration stimulus. It is preferable to set the magnitude of pressing force from 1 to 2 kgf (kilogram-weight) and to set the one time continuous imparting time from 20 to 40 seconds for the patterns of the second stimulus imparting device 15 and the fourth stimulus imparting device 17 among the first stimulus patterns when the stimulus imparted is the stimulus of pressing force.

The optimum frequency is derived from the experimental work. The experimental result is shown in FIG. 9. FIG. 9 indicates the experimental result that a vibration stimulus is imparted to the origin side (rib) tendon (including muscle tendon junction) of upper side fiber among the tendon portion of latissimus dorsi when a person in test is at the first drowsiness level where the person does not feel drowsy. The horizontal axis indicates the time between the imparting stimulus is started and a certain time period passed therefrom and the vertical axis indicates the level of drowsiness.

The time for continuously imparting stimulus is fixed to thirty seconds and the drowsiness level is measured by changing the frequency from 50 Hz to 120 Hz. The drowsiness level is defined by self-assessment of the subject person or may be defined by measurement. The white square indicates the change of drowsiness level when the stimulus is imparted for thirty seconds continuously with the frequency of 50 Hz, the diamond indicates the change of drowsiness level when the stimulus is imparted for thirty seconds continuously with the frequency of 80 Hz, the circle indicates the change of drowsiness level when the stimulus is imparted for thirty seconds continuously with the frequency of 100 Hz and the triangle indicates change of drowsiness level when the stimulus is imparted for thirty seconds continuously with the frequency of 120 Hz. As apparent from the experimental result, the optimum frequency is 100 Hz. From the peak frequency of 100 Hz, the drowsiness level goes to the drowsiness side when the frequency goes down or up from this peak frequency.

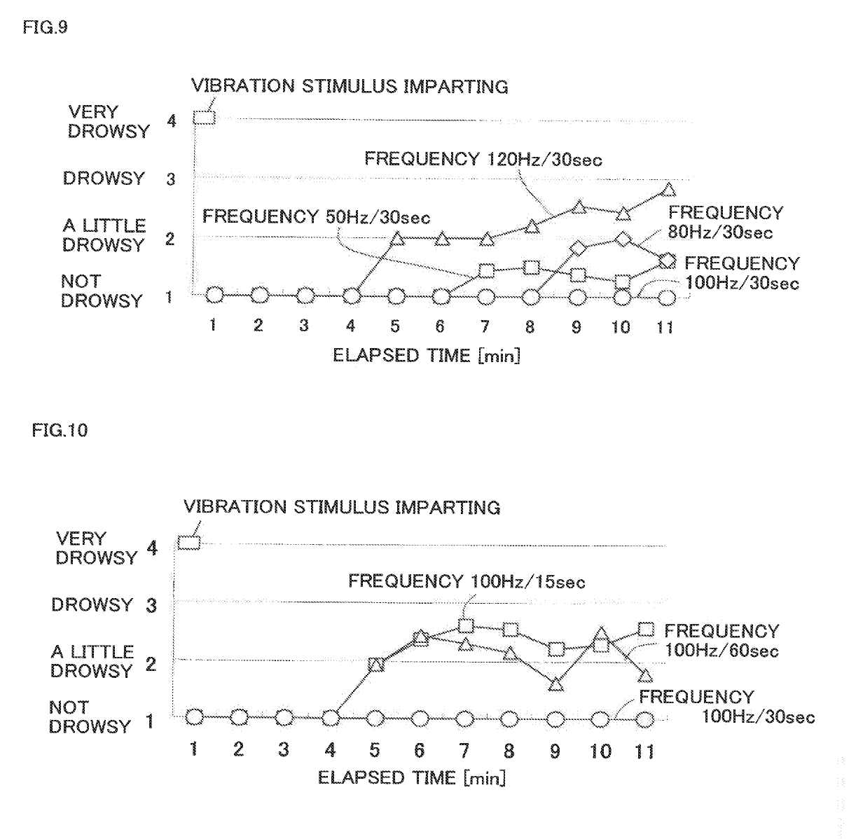

The optimum one time continuous imparting time is derived from the experimental work as well. The experimental result is shown in FIG. 10. FIG. 10 indicates the experimental result that a vibration stimulus is imparted to the origin side (rib) tendon (including muscle tendon junction) of upper side fiber among the tendon portion of latissimus dorsi when a person in test is at a first drowsiness level where the person does not feel drowsy. The horizontal axis indicates the time between the imparting stimulus is started and a certain time period passed therefrom and the vertical axis indicates the level of drowsiness.

The frequency is fixed to 100 Hz and the drowsiness level is measured by changing the continuous imparting time from 15 to 60 seconds. The drowsiness level is defined by self-assessment of the subject person or may be defined by measurement. The white square indicates the change of drowsiness level when the stimulus is imparted for fifteen seconds continuously with the frequency of 100 Hz, the circle indicates the change of drowsiness level when the stimulus is imparted for thirty seconds continuously with the frequency of 100 Hz and the triangle indicates change of drowsiness level when the stimulus is imparted for sixty seconds continuously with the frequency of 100 Hz. As apparent from the experimental result, the optimum continuous imparting time is thirty seconds. The drowsiness level goes up to the drowsiness side from an early time in the case of fifteen seconds imparting time or sixty seconds imparting time.

As explained above, the stimulus imparted with the frequency of 100 Hz and with one time continuous imparting time of thirty seconds has the highest awakening effect. This effect can be achieved not only at the tendon of the latissimus dorsi but also at the tendon of gluteus medius muscle (particularly, the origin side tendon (including muscle tendon junction)).

The first stimulus pattern may be the stimulus pattern in which the vibration that vibrates all stimulus imparting device groups 12R, 12L, 13R and 13L at the same time (all vibrations) is repeatedly imparted or may be the stimulus pattern in which the vibration that vibrates only the right side first stimulus imparting device 14R and the left side first stimulus imparting device 14L at the same time (latissimus dorsi vibration) is repeatedly imparted, or further may be the stimulus pattern in which the vibration that vibrates only the right side third stimulus imparting device 16R and the left side third stimulus imparting device 16L at the same time (gluteus medius muscle vibration) is repeatedly imparted. It may also be the stimulus pattern in which all vibrations at the first time, the latissimus dorsi vibration at the second time and the gluteus medius muscle vibration at the third time are imparted and thereafter this order imparting stimulus is repeated. The order of the stimulus imparting is not limited to the above order. Further, the stimulus pattern may include the vibration that vibrates only the right side stimulus imparting device groups 12R and 13R (right side vibration) and the vibration that vibrates only the left side stimulus imparting device groups 12L and 13L (left side vibration).

The second stimulus pattern determining portion 43b determines the second stimulus pattern which eliminates the drowsiness of the driver M when the drowsiness eliminating judging portion 42 judges that it is necessary to eliminate the drowsiness of the driver M. The second stimulus pattern is basically the same with the first stimulus pattern.

The driving control portion 44 drives at least one of the first stimulus imparting device group 12 and the second stimulus imparting device group 13 based on each stimulus pattern determined by each stimulus pattern determining portion 43a and 43b to thereby impart stimulus to the driver M.

When it is judged that the drowsiness of the driver M is necessary to be eliminated, the driving control portion 44 drives the stimulus imparting device groups 12R, 12L, 13R and 13L based on the first stimulus pattern to impart the stimulus to the driver M to perform the drowsiness eliminating support which eliminates the drowsiness of the driver M. Further, when the driver M itself demands the stimulus (when the driver M turns the stimulus imparting switch 33 on), the driving control portion 44 drives the stimulus imparting device groups 12R, 12L, 13R and 13L based on the first stimulus pattern to impart the stimulus to the driver M to perform the drowsiness eliminating support which eliminates the drowsiness of the driver M.

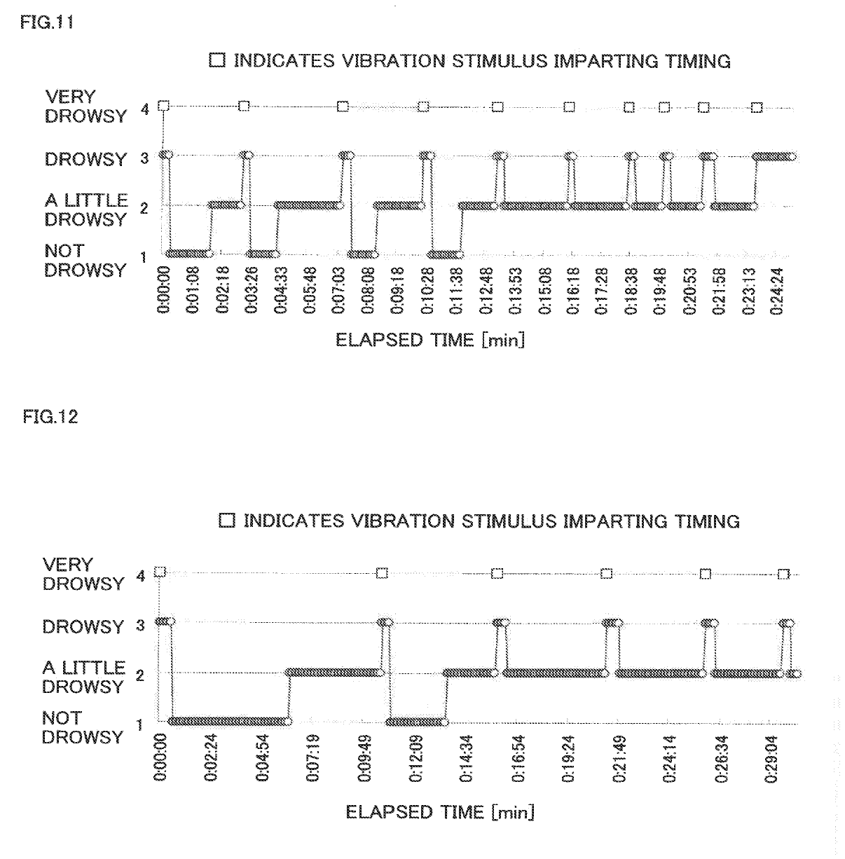

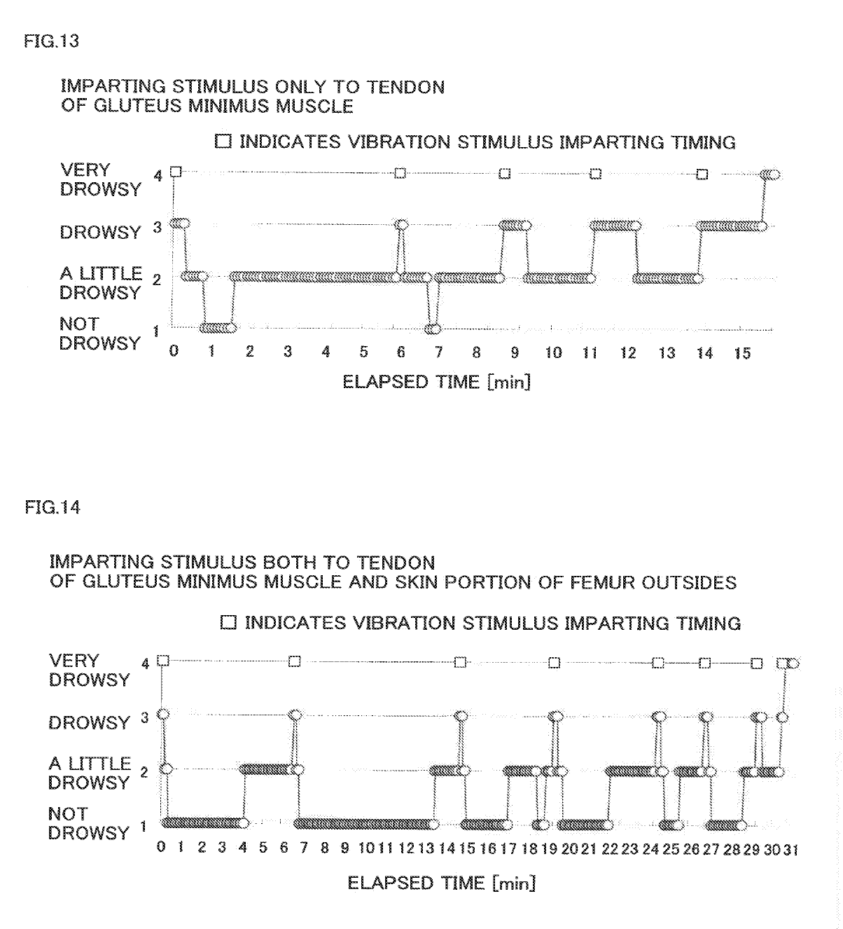

The effect of the stimulus imparted to the driver M will be explained hereinafter. The drowsiness eliminating effect among the effects when the stimulus is imparted to particular human body portions is shown in FIG. 11 and FIG. 12. FIG. 11 indicates the experimental result that a vibration stimulus is imparted to the origin side (rib) tendon (including muscle tendon junction) of upper side fiber latissimus dorsi of a person in test when the person in test is at a third drowsiness level where the person feels drowsy. The imparted vibration stimulus is a stimulus with the frequency of 100 Hz with thirty second continuously imparted time. The white square indicates the timing of vibration stimulus imparting and this timing agrees with the timing that the person in test has reached to the third drowsiness level at which the person in test feels drowsy. The timing immediately after the vibration stimulus is imparted indicates the non-drowsy first drowsiness level. Thus, from this experiment, it is confirmed that imparting the vibration stimulus has a high effect in drowsiness elimination.

FIG. 12 indicates the experimental result that a vibration stimulus is imparted to the origin side tendon (including muscle tendon junction) of gluteus medius muscle of a person in test when the person in test is at a third drowsiness level where the person feels drowsy. The vibration stimulus imparted is a stimulus with the frequency of 100 Hz with thirty second continuously imparted time. The white square indicates the timing of vibration stimulus imparting and this timing agrees with the timing that the person in test has reached to the third drowsiness level at which the person in test feels drowsy. The timing immediately after the vibration stimulus is imparted indicates the non-drowsy first drowsiness level. Thus, from this experiment, it is confirmed that imparting the vibration stimulus has a high effect in drowsiness elimination. Further, the drowsiness eliminating effect (drowsiness generation suppression effect) of imparting stimulus to the tendon of gluteus medius muscle is longer maintained than the drowsiness eliminating effect of imparting stimulus to the tendon of latissimus dorsi.

Further, the effect of imparting stimulus to both particular human body portion (such as for example, tendon of latissimus dorsi and tendon of gluteus medius muscle) and a skin portion (inferior angle of scapula to the armpit portion and the femur outside portions) in the vicinity of skeletal muscle associated with the motion illusion induced at the supplementary motor area activated by the stimulus to the particular human body portion will be explained with reference to FIGS. 13 through 15. It is noted that FIGS. 13 through 15 show the latissimus dorsi and gluteus medius muscle and the inferior angle of scapula to the armpit portion is omitted.

FIG. 13 indicates the experimental result that a vibration stimulus is imparted to only the tendon of gluteus medius muscle of a person in test when the person in test is at a third drowsiness level where the person feels drowsy. The vibration stimulus imparted is a stimulus with the frequency of 100 Hz with thirty second continuously imparted time. The white square indicates the timing of vibration stimulus imparting and this timing agrees with the timing that the person in test has reached to the third drowsiness level at which the person in test feels drowsy. When the stimulus is imparted, the drowsiness level goes down to the second level where the person feels slightly drowsy or the first level where the person feels non-drowsy. In other words, the drowsiness has been eliminated. However, such stimulus is imparted several times, even such stimulus is imparted the drowsiness level does not go down the drowsiness is not eliminated. The drowsiness level goes up to the fourth drowsiness level at which level, a person feels very drowsy. The time from the first imparting stimulus to the time when the drowsiness level reaches to the fourth drowsiness level is about fifteen minutes. Within the fifteen minutes, the first drowsiness level of non-drowsy is about one minute, the second drowsiness level of slightly drowsy is about twelve minutes and the third drowsiness level of drowsy is about two minutes.

On the contrary, FIG. 14 indicates the experimental result that the vibration stimulus is imparted to both tendon portion of the gluteus medius muscle and the skin portion of the femur outside portions when the person in test is at a third drowsiness level where the person feels drowsy. The vibration stimulus imparted to the tendon portion of the gluteus medius muscle is a stimulus with the frequency of 100 Hz with thirty second continuously imparted time. The vibration stimulus imparted to the skin portion of the femur outside portion is a stimulus with the frequency of 50 Hz with thirty second continuously imparted time. The white square indicates the timing of vibration stimulus imparting and this timing agrees with the timing that the person in test has reached to the third drowsiness level at which the person in test feels drowsy. When the stimulus is imparted, the drowsiness level goes down to the second level where the person feels slightly drowsy or the first level where the person feels non-drowsy. In other words, the drowsiness has been eliminated. However, such stimulus is imparted several times, even such stimulus is imparted the drowsiness level does not go down the drowsiness is not eliminated. The drowsiness level goes up to the fourth drowsiness level at which level, a person feels very drowsy. The time from the first imparting stimulus to the time when the drowsiness level reaches to the fourth drowsiness level is about thirty minutes. Within the thirty minutes, the first drowsiness level of non-drowsy is about eighteen minutes, the second drowsiness level of slightly drowsy is about eleven minutes and the third drowsiness level of drowsy is about one minute.

Thus, the time of the third drowsiness level or below the third drowsiness level from the stimulus imparting starting, which is one parameter that indicates the drowsiness eliminating effect, is longer in the case of the stimulus imparting to both tendon portion of the gluteus medius muscle and the skin portion of the femur outside portion than in the case of the stimulus imparting only to the tendon of gluteus medius muscle. Further, the ratio of the time the first drowsiness level or the second drowsiness level occupies is larger than the ratio of the time the third drowsiness level or below the third drowsiness level occupies from the stimulus imparting starting, which is one parameter that indicates the drowsiness eliminating effect.

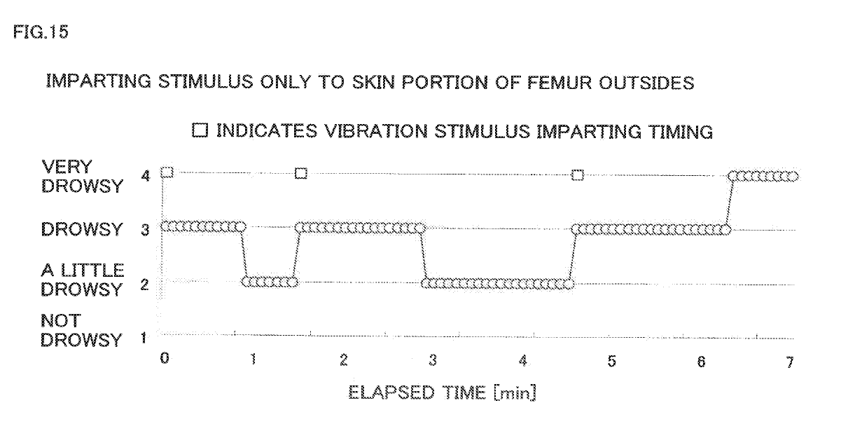

It is noted that as a reference data, FIG. 15 indicates the experimental result that a vibration stimulus is imparted to only to the skin portion of the femur outside portion of a person in test when the person is in the third drowsiness level. The vibration stimulus imparted to the skin portion of the femur outside portion is the stimulus which frequency is 50 Hz and the time of imparting stimulus continuously is thirty seconds. The white square indicates the timing of vibration stimulus imparting and this timing agrees with the timing that the person in test reached to the third drowsiness level at which the person in test feels drowsy. When the vibration stimulus is imparted, the drowsiness level goes down to the second drowsiness level at which the person in test feels slightly drowsy but does not go down to the first drowsiness level which is a non-drowsy level. In other words, the drowsiness has been somewhat eliminated. However, when the vibration stimulus is imparted a number of times, even the vibration stimulus is imparted, the drowsiness cannot be eliminated. At this timing, the drowsiness level does not go down but goes up to the fourth drowsiness level. The time from the first imparting stimulus to the time when the drowsiness level reaches to the fourth drowsiness level is about six minutes. Within the six minutes, the first drowsiness level of non-drowsy is zero minute, the second drowsiness level of slightly drowsy is about two minutes and the third drowsiness level of drowsy is about four minutes.

It is noted that FIGS. 13 through 15 show the gluteus medius muscle and femur outside portion and the latissimus dorsi and the inferior angle of scapula to the armpit portion are omitted. However, similar to the gluteus medius muscle and femur outside portion and the latissimus dorsi, similar tendency of the awakening effect for the latissimus dorsi and the inferior angle of scapula to the armpit portion is expected.

As shown in FIGS. 4 and 5, the drowsiness generation eliminating effect among the effects when the stimulus is imparted to particular human body portions is that the drowsiness level at latissimus dorsi and the gluteus medius muscle is maintained to the first drowsiness level of non-drowsy for relatively a long period of time. Accordingly, the drowsiness generation suppression effect is high when the stimulus is imparted to the latissimus dorsi and the gluteus medius muscle, i.e., a high awakening effect can be maintained.

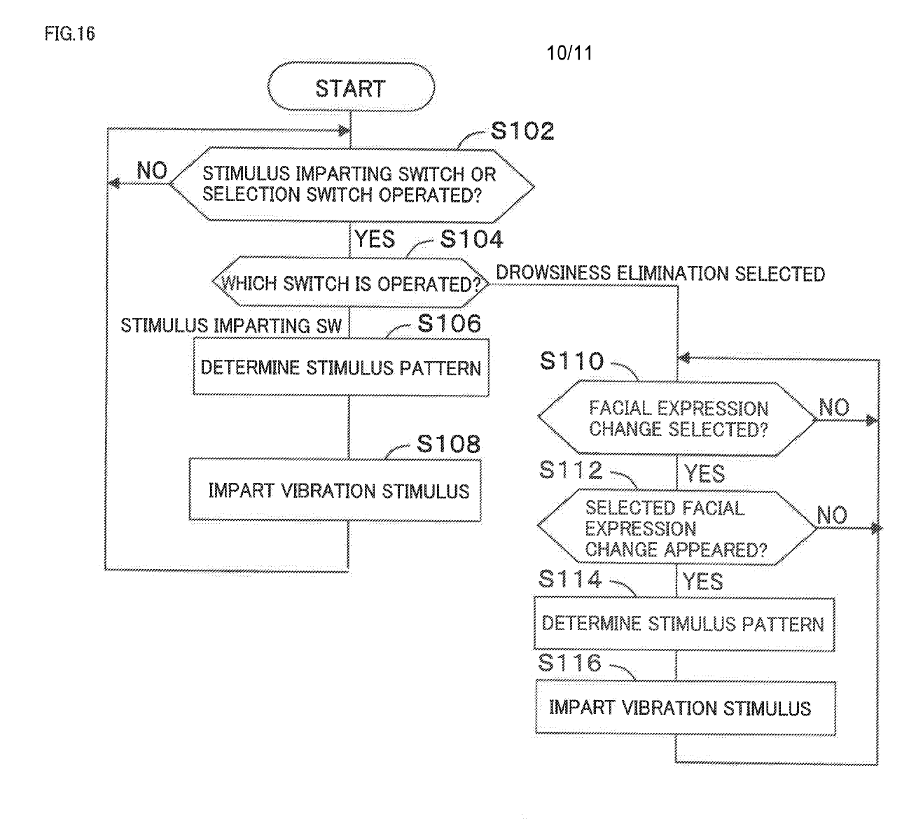

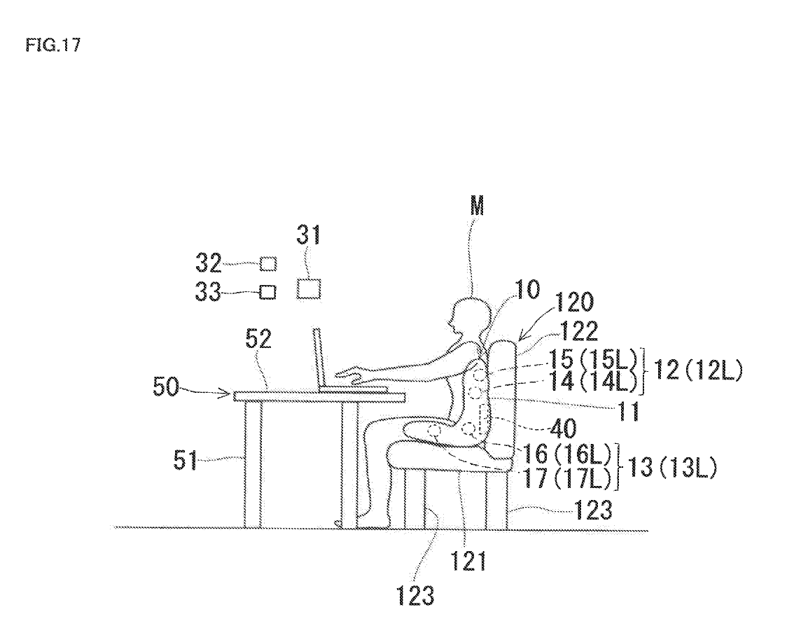

The operation of above structured drowsiness eliminating device 10 will be explained hereinafter with reference to the flowchart shown in FIG. 16.

The control device 40 obtains and judges the switch state of the selection switch 32 and the stimulus imparting switch 33 at the step S102, as similar to the above explained switch state obtaining/judging portion 41. In other words, the control device 40 judges whether the selection switch 32 and the stimulus imparting switch 33 are operated or not.

When the selection switch 32 and the stimulus imparting switch 33 are not operated, the control device 40 repeatedly executes the processing of the step S102. When the selection switch 32 and the stimulus imparting switch 33 are operated, then the control device 40 advances the program to the step S104 and thereafter.

At the step S104, the control device 40 judges the operated switch as similar to the above explained switch state obtaining/judging portion 41. When the stimulus imparting switch 33 is ON, the control device 40 advances the program to the step S106 or when the selection that the drowsiness eliminating support is necessary is selected, the control device 40 advances the program to the step S110.

When the stimulus imparting switch 33 is ON (i.e., when the driver M itself demands the stimulus imparting), the control device 40 determines the first stimulus pattern at the step S106, as similar to the first stimulus pattern determining portion 43a. Then, at the step S108, the control device 40 controls to drive each of the stimulus imparting device groups 12R, 12L, 13R and 13L based on the first stimulus pattern as similar to the above explained driving control portion 44 thereby to impart stimulus to the driver M to support drowsiness elimination which eliminates the drowsiness of the driver M.

The control device 40 judges whether or not the elimination of the drowsiness of the driver M seated on the vehicle seat (drowsiness elimination) is necessary at the steps S104, S110 and S112 (drowsiness eliminating judgement), as similar to the above explained drowsiness eliminating judging portion 42 (judging portion). In detail, the control device 40 judges that it is necessary to eliminate a drowsiness of the driver M, when expression change of the judgement subject is selected (determined) (“YES” at the step S110) and the selected expression change appears (“YES” at the step S112).

When it is judged that it is necessary to eliminate a drowsiness of the driver M, the control device 40 determines the second stimulus pattern at the step S114, as similar to the above explained second stimulus pattern determining portion 43b. Then, at the step S116, the control device 40 controls to drive each stimulus imparting device group 12R, 12L, 13R and 13L based on the second stimulus pattern, as similar to the above explained driving control portion 44, thereby to impart stimulus to the driver M. Thus, the drowsiness eliminating support for the driver M is performed.

As apparent from the first embodiment, the drowsiness eliminating device according to the first embodiment includes at least one of a first stimulus imparting device group 12 including a first stimulus imparting device 14 provided on a vehicle seat 20 (chair) such that the tendon of latissimus dorsi of a driver M (user) of the vehicle seat 20 faces when the driver M sits on the vehicle seat 20 and imparting stimulus to the tendon of latissimus dorsi of the driver M and a second stimulus imparting device 15 provided on the vehicle seat 20 facing a first skin portion when the driver M sits on the vehicle seat 20 and imparting the stimulus to the first skin portion of the driver M which is a skin part covering a skeletal muscle which is associated with a first motion illusion induced at a supplementary motor area activated by the stimulus to the tendon of latissimus dorsi and which is a cooperative muscle of the latissimus dorsi and a second stimulus imparting device group 13 including a third stimulus imparting device 16 provided on the vehicle seat 20 such that the tendon of gluteus medius muscle of the driver M of the vehicle seat 20 faces when the driver M sits on the vehicle seat and imparting the stimulus to the tendon of gluteus medius muscle of the driver M and a fourth stimulus imparting device 17 provided on the vehicle seat facing a second skin portion when the driver M sits on the vehicle seat 20 and imparting the stimulus to the second skin portion of the driver M which is a skin part covering a skeletal muscle which is associated with a second motion illusion induced at a supplementary motor area activated by the stimulus to the tendon of the gluteus medius muscle and which is a cooperative muscle of the gluteus medius muscle and a drive control device 40 which includes a driving control portion 44 which drive-controls said at least one of the first stimulus imparting device group and the second stimulus imparting device group to impart a stimulus to the driver M of the vehicle seat 20.

According to the configuration, the driving control portion 44 imparts stimulus to the tendon of latissimus dorsi of the driver M by the first stimulus imparting device 14 and to the first skin portion of the driver M by the second stimulus imparting device 15, and/or the driving control portion 44 imparts stimulus to the tendon of gluteus medius muscle of the driver M by the third stimulus imparting device 16 and to the second skin portion of the driver M by the fourth stimulus imparting device 17. Accordingly, in addition to properly stimulating tendon of latissimus dorsi and/or gluteus medius muscle which are the body parts highly effective to awaken the driver M, it is possible to properly stimulate a first skin portion which is a skin part covering the skeletal muscle which is associated with a first motion illusion which is induced at the supplementary motor area activated by the stimulus to the tendon of latissimus dorsi and which is a cooperative muscle of the latissimus dorsi of the driver M of the vehicle seat 20 and/or to properly stimulate a second skin portion which is a skin part covering the skeletal muscle which is associated with a second motion illusion which is induced at the supplementary motor area activated by the stimulus to the tendon of gluteus medius muscle and which is a cooperative muscle of the gluteus medius muscle of the driver M. Thus, by simulating two portions which awakening effects are mutually correlated to thereby achieve a higher awakening effect. Accordingly, the drowsiness eliminating device 10 can properly stimulate the portions where the awakening effect is high.

Further, the strength of the stimulus imparted from the second stimulus imparting device 15 is set to be smaller than the strength from the first stimulus imparting device 14 and the strength of the stimulus imparted from the fourth stimulus imparting device 17 is set to be smaller than the strength from the third stimulus imparting device 16.

According to the configuration, the drowsiness eliminating device 10 can effectively achieve the awakening effect by properly imparting the stimulus depending on the place or portion where the stimulus is imparted.

Thus, when the stimulus imparted from the second stimulus imparting device 15 and the fourth stimulus imparting device 17 is the vibration stimulus, the frequency of the vibration can be set to the frequency from 40 Hz to 60 Hz.

According to this configuration, the drowsiness eliminating device 10 can impart more appropriate stimulus to a portion which is highly effective in awakening effect.

When the stimulus imparted from the second stimulus imparting device 15 and the fourth stimulus imparting device 17 is the pressing force stimulus, the magnitude of the pressing force can be set to the value from 1 to 2 kgf (kilogram-force).

According to this configuration, the drowsiness eliminating device 10 can impart more appropriate stimulus to a portion which is highly effective in awakening.

Further, the drowsiness eliminating device includes the drowsiness eliminating judging portion 42 (judging portion) which judges whether or not it is necessary to eliminate the drowsiness of the driver M sitting on the vehicle seat 20 and the first stimulus pattern determining portion 43a which determines the first stimulus pattern for eliminating the drowsiness when the drowsiness eliminating judging portion 42 judges that it is necessary to eliminate the drowsiness of the driver M, wherein the driving control portion 44 imparts the stimulus to the driver M by drive-controlling at least one of the first stimulus imparting device group 12 and the second stimulus imparting device group 13 to impart a stimulus to the driver M based on the first stimulus pattern determined by the first stimulus pattern determining portion 43a. According to this configuration, the drowsiness eliminating device 10 can impart more appropriate stimulus to a portion which is highly effective in awakening effect.

The “chair” corresponds to the vehicle seat 20 installed in the vehicle A and the “user” corresponds to the driver M.

According to this configuration, the drowsiness eliminating device 10 which can properly impart the stimulus to a portion which is highly effective to the awakening effect can be applicable to the vehicle A and as a result, the drowsiness of the driver M can be sufficiently eliminated.

### Second Embodiment

Next, the second embodiment will be explained with reference to FIG. 17. The second embodiment is different from the first embodiment (applied to the vehicle) in applying the application to a furniture. In detail, the chair corresponds to a chair 120, instead of the vehicle seat 20. The control device 40 is housed in the main body 11 of the drowsiness eliminating device 10.

The chair 120 is a chair on which the user M sits and is formed by a seat cushion 121 and a seat back 122 and a plurality of legs 123. The chair 120 is used with a desk 50 as a set. The desk 50 is formed by a board portion 52 and a plurality of leg portions 51. A user monitor 31, a selection switch 32 and a stimulus imparting switch 33 are arranged in the vicinity of the desk 50. Other configurations are the same with those of the first embodiment and the explanation thereof will be omitted with the same reference numerals.

The drowsiness eliminating device according to the second embodiment configured as explained above includes, similar to the first embodiment, at least one of a first stimulus imparting device group 12 including a first stimulus imparting device 14 provided on a chair 120 such that the tendon of latissimus dorsi of a user M of the chair 120 faces when the user M sits on the chair 120 and imparting a stimulus to the tendon of latissimus dorsi of the user M and a second stimulus imparting device 15 provided on the chair 120 facing a first skin portion when the user M sits on the chair 120 and imparting the stimulus to the first skin portion of the user M which is a skin part covering a skeletal muscle which is associated with a first motion illusion induced at a supplementary motor area activated by the stimulus to the tendon of latissimus dorsi and which is a cooperative muscle of the latissimus dorsi and a second stimulus imparting device group 13 including a third stimulus imparting device 16 provided on the chair 120 such that the tendon of gluteus medius muscle of the user M of the chair 120 faces when the user M sits on the chair 120 and imparting a stimulus to the tendon of gluteus medius muscle of the user M and a fourth stimulus imparting device 17 provided on the chair 120 facing a second skin portion when the user M sits on the chair 120 and imparting the stimulus to the second skin portion of the user M which is a skin part covering a skeletal muscle which is associated with a second motion illusion induced at a supplementary motor area activated by the stimulus to the tendon of gluteus medius muscle and which is a cooperative muscle of the gluteus medius muscle and a drive control device 40 which includes a driving control portion 44 which drive-controls at least one of the first stimulus imparting device group and the second stimulus imparting device group to impart a stimulus to the user M of the chair 120.

According to the configuration, the driving control portion 44 imparts stimulus to the tendon of latissimus dorsi of the user M by the first stimulus imparting device 14 and to the first skin portion of the user M by the second stimulus imparting device 15, and/or the driving control portion 44 imparts stimulus to the tendon of gluteus medius muscle of the user M by the third stimulus imparting device 16 and to the second skin portion of the user M by the fourth stimulus imparting device 17. Accordingly, in addition to properly stimulating tendon of latissimus dorsi and/or gluteus medius muscle which are the body parts highly effective to awaken the user M, it is possible to properly stimulate a first skin portion which is a skin part covering the skeletal muscle which is associated with a first motion illusion which is induced at the supplementary motor area activated by the stimulus to the tendon of latissimus dorsi and which is a cooperative muscle of the latissimus dorsi of the user M of the chair 120 and/or to properly stimulate a second skin portion which is a skin part covering the skeletal muscle which is associated with a second motion illusion which is induced at the supplementary motor area activated by the stimulus to the tendon of gluteus medius muscle and which is a cooperative muscle of the gluteus medius muscle of the user M. Thus, by simulating two portions which awakening effects are mutually correlated to thereby achieve a higher awakening effect. Accordingly, the drowsiness eliminating device 10 can properly stimulate the portions where the awakening effect is high.

According to the embodiments explained above, the first stimulus imparting device group 12 and the second stimulus imparting device group 13 are both arranged. However, it may be configured to have only one of the first stimulus imparting device group 12 and the second stimulus imparting device group 13.

Further, the driving control portion 44 is configured to drive both of the first stimulus imparting device group 12 and the second stimulus imparting device group 13. However, it may be configured to drive either one of the first stimulus imparting device group 12 and the second stimulus imparting device group 13.

Further, instead of using stimulus imparting switch 33, a voice recognizing device is provided to request a stimulus by sound.

### REFERENCE SIGNS LIST

10; drowsiness eliminating device, 11; main body, 12; first stimulus imparting device group, 13; second stimulus imparting device group, 14; first stimulus imparting device, 14R; right side first stimulus imparting device, 14L; left side first stimulus imparting device, 15; second stimulus imparting device, 15R; right side second stimulus imparting device, 15L; left side second stimulus imparting device, 16; third stimulus imparting device, 16R; right side third stimulus imparting device, 16L; left side third stimulus imparting device, 17; fourth stimulus imparting device, 17R; right side fourth stimulus imparting device, 17L; left side fourth stimulus imparting device, 20; vehicle seat (chair), 31; driver monitor, user monitor, 32; selection switch, 33; stimulus imparting switch, 40; control device, 41; switch state obtaining/judging portion, 42; drowsiness eliminating judging portion, 43a; 43b; each stimulus pattern determining portion, 44; driving control portion, 120; chair, A; vehicle, M; driver/user

1. A drowsiness eliminating device comprising:
at least one of:
a first stimulus imparting device group including:
a first stimulus imparting device provided on a chair such that a tendon of latissimus dorsi of a user of the chair faces when the user sits on the chair and imparting a stimulus to the tendon of latissimus dorsi of the user; and
a second stimulus imparting device provided on the chair facing a first skin portion when the user sits on the chair and imparting a stimulus to the first skin portion of the user which is a skin part covering a skeletal muscle which is a cooperative muscle of the latissimus dorsi and is associated with a first motion illusion induced at a supplementary motor area activated by the stimulus to the tendon of latissimus dorsi; and
a second stimulus imparting device group including:
a third stimulus imparting device provided on the chair such that a tendon of gluteus medius muscle of the user of the chair faces when the user sits on the chair and imparting a stimulus to the tendon of gluteus medius muscle of the user; and
a fourth stimulus imparting device provided on the chair facing a second skin portion when the user sits on the chair and imparting a stimulus to the second skin portion of the user which is a skin part covering a skeletal muscle which is associated with a second motion illusion induced at a supplementary motor area activated by the stimulus to the tendon of gluteus medius muscle and which is a cooperative muscle of the gluteus medius muscle; and
a drive control device which includes a driving control portion which drive-controls said at least one of the first stimulus imparting device group and the second stimulus imparting device group to impart a stimulus to the user of the chair.

2. The drowsiness eliminating device according to claim 1, wherein
a magnitude of the stimulus imparted from the second stimulus imparting device is set to be smaller than a magnitude of the stimulus imparted from the first stimulus imparting device and
a magnitude of the stimulus imparted from the fourth stimulus imparting device is set to be smaller than a magnitude of the stimulus imparted from the third stimulus imparting device.

3. The drowsiness eliminating device according to claim 1, wherein
when the stimulus imparted from the second stimulus imparting device and the fourth stimulus imparting device is a vibration stimulus, a frequency of the vibration is set from 40 Hz to 60 Hz.

4. The drowsiness eliminating device according to claim 1, wherein
when the stimulus imparted from the second stimulus imparting device and the fourth stimulus imparting device is a pressing force stimulus, a magnitude of the pressing force is set to a value from 1 to 2 kgf.

5. The drowsiness eliminating device according to claim 1, further comprising:
a judging portion for judging whether or not it is necessary to eliminate a drowsiness of the user sitting on the chair; and
a first stimulus pattern determining portion for determining a first stimulus pattern for eliminating the drowsiness of the user when the judging portion judges that it is necessary to eliminate the drowsiness of the user, wherein
the driving control portion imparts the stimulus to the user by drive-controlling said at least one of the first stimulus imparting device group and the second stimulus imparting device group to impart the stimulus to the user based on the first stimulus pattern determined by the first stimulus pattern determining portion.

6. The drowsiness eliminating device according to claim 1, wherein
the chair is a vehicle seat installed in a vehicle and the user is a driver of the vehicle.
